(19) United States
(12) Patent Application Publication
(10) Pub. No.: US 2024/0350245 A1
(43) Pub. Date: Oct. 24, 2024

(54) RADIATION DEVICE FOR ACTIVATING POLYMERIZABLE DENTAL MATERIALS

(71) Applicant: KULZER GMBH, Hanau (DE)

(72) Inventor: Andreas UTTERODT, Neu Anspach (DE)

(21) Appl. No.: 18/637,795

(22) Filed: Apr. 17, 2024

(30) Foreign Application Priority Data
Apr. 18, 2023 (DE) 102023109682.6

(51) Int. Cl. A61C 13/15; A61K 6/884; G02B 26/02; G02B 26/08; H05B 47/105

(52) U.S. Cl. CPC A61C 19/003; G02B 26/023; G02B 26/0816; H05B 47/105; A61K 6/884

(57) ABSTRACT

A radiation device comprises: a housing comprising: a base portion; a head portion connected to the base portion; a radiation emission window in the head portion; a radiation source arranged in the region of the base portion in the interior of the housing for emitting electromagnetic radiation, the radiation source having an emission intensity maximum at a wavelength in the range from 350 to 600 nm; and a mirror arranged in the region of the head portion in the interior of the housing. A radiation path extends between the radiation source and the mirror. Electromagnetic radiation emitted by the radiation source arrives at the mirror via the radiation path. The electromagnetic radiation arriving at the mirror via the radiation path is reflected by the mirror, such that the reflected electromagnetic radiation exits the housing through the radiation emission window.

### FIELD OF THE INVENTION

The invention relates to a radiation device for use in activating polymerizable dental materials, and to a method for operating such a radiation device. Also disclosed is the use of a corresponding radiation device for activating polymerizable dental materials in the manufacture and/or processing of dental restorations.

### BACKGROUND

In modern dentistry, resin-based dental materials are regularly used for the production of dental restorations, such as fillings. These dental materials can be polymerized, in particular polymerized using activation by light, in the state intended for application, they have not yet reached their final degree of curing. The corresponding dental materials can be inserted, before curing, into a cavity created in a tooth, for example, where they can be optimally adapted to the shape of the cavity, so that the defect can be reliably repaired. The corresponding dental materials are only cured after application, which means that cross-linking is triggered between the components contained in the dental material, resulting in curing and thus a change in key physico-chemical parameters such as hardness and strength.

Even though various mechanisms for curing such polymerizable resins are known in principle in the field of polymer chemistry, for example by thermal activation or the use of a separate hardener component, radiation-based curing is particularly important in the dental field, as it is associated with considerable application-related advantages for the intended use in a patient's mouth.

Corresponding radiation-curable dental materials, in which polymerization can be initiated by activation with blue or ultraviolet light, for example, are commercially available from numerous manufacturers. Suitable photoinitiators are used in corresponding radiation-curable dental materials, which, when irradiated with electromagnetic radiation of a certain wavelength, cause the polymerization of the dental material, for example by the decomposition of the photoinitiator forming radical components which can lead to a radical polymerization of unsaturated compounds in the dental material, in particular (meth)acrylate compounds.

To ensure sufficient cross-linking of the introduced material, in particular over the entire depth of the filled cavity, and to ensure good reproducibility, high demands are placed on the light intensity emitted by the radiation device, which should ideally also be matched to the dental material used and the size of the cavity in order to achieve optimum polymerization quality. Radiation devices suitable for use in the curing of corresponding dental materials, which are also referred to by those skilled in the art as light devices, are known from the prior art and are commercially available from various manufacturers. Light devices for the emission of electromagnetic radiation with a wavelength in the blue range are particularly common for dental applications.

In the past, halogen lamps with a suitable light filter, e.g., blue light filters, were generally used as a radiation source in corresponding lighting devices. In the course of ongoing technical development, however, LEDs with suitable emission wavelength ranges are primarily used today.

Typical commercially available devices differ in terms of their design and optics, but their construction is usually subject to the application requirement that the corresponding light must be emitted in the mouth in an ergonomic manner. In other words, the electromagnetic radiation required to cure the polymerizable dental materials must be able to be applied efficiently and comfortably in the patient's mouth, especially in the region of teeth and/or the positions of the cavities to be filled that are difficult to access.

In practice, the exposure site in dentistry is rarely freely accessible, and requires good access with the radiation device between the teeth in poorly accessible areas, and possibly at unfavorable angles. In order to deliver the light required for polymerization with the necessary quality, i.e., suitable wavelength and intensity, to the application site as required, curved light guides are often used in the state of the art. In this approach, the radiation source is regularly positioned in the rear part of the usually elongated radiation device, which is advantageous in terms of control, energy supply and, in particular, heat dissipation from the radiation source used. Via a suitable radiation path, the electromagnetic radiation generated by the radiation source is then introduced into a curved light guide that forms the upper end of the elongated radiation device, which is inserted into the patient's mouth for use.

The light guide is curved in order to deflect the path of the electromagnetic radiation in such a way that even challenging angles in the oral cavity can be realized. Even if acceptable results can be achieved with this type of radiation device in the state of the art, the corresponding design is sometimes perceived as disadvantageous in the field of the applied technology. For example, in many cases the curved light guide at the end of the radiation device means that the part of the radiation device inserted into the oral cavity is relatively large, i.e., has a large height, which can make handling more difficult and can be perceived as uncomfortable by the patient. In addition, a strong bend in the light guide is usually desired, as a small radius of curvature is preferred. However, this can lead to an undesirable reduction in light intensity, which has a negative impact on energy efficiency and/or the maximum achievable radiant power.

To circumvent the problems described above, an alternative design was proposed, which was made possible by the use of miniaturized LED components as the radiation source. In this case, the semiconductors of the LED components are implemented directly below a lateral light emission window, and are therefore located directly in the part of the radiation device that is inserted into the patient's mouth and brought close to the cavity. In an advantageous way, larger emission angles of, for example, 90° (in relation to the longitudinal axis of the radiation device) can be achieved, and a lower overall height is also advantageously achieved, which is substantially determined by the size of the electronics used. Even though such constructions are seen as advantageous in many respects, they are also considered disadvantageous in certain aspects. Sufficient heat dissipation during operation of the semiconductor components is technically difficult in many cases, especially with high emission power. Another problem is the usually undesirably high divergence of the emitted light, so that the desired high light intensity is usually only achieved at a close distance from the light emission window, and the desired homogeneity of the radiation power is difficult to achieve.

Compensating for the problems described above usually requires technically complex solutions, such as a sophisticated cooling system for heat dissipation or the use of laser diodes that can also generate the necessary light intensity at a greater distance and are therefore also suitable for curing dental materials in deep cavities. However, these technical solutions, as well as the basic design using miniaturized LED components, are regularly associated with high demands on the components used and their complexity. This increases the costs for the components used as well as the manufacturing costs for the production of the corresponding lighting devices, so that these technically advantageous solutions cannot be produced in a time and cost-efficient manner in most cases.

In addition to the aspects described above, there are also certain reservations with regard to the durability of the corresponding radiation devices. For hygienic reasons, the parts of the radiation device that are inserted into the patient's mouth during use must be cleaned or sterilized particularly thoroughly on a regular basis. In practice, against this background, it is therefore often desirable to work with multi-part radiation devices that are equipped with an exchange mechanism so that the parts introduced into the oral cavity can be exchanged quickly and easily in order to be able to produce a fresh radiation device without delay after the treatment of a first patient by exchanging the contaminated components, while the previously used component undergoes the intended preparation procedures. In the design described above, in which the radiation source and large parts of the necessary electronics, and possibly a complex cooling system, are located within the portion of the radiation device that would have to be replaced and refurbished, the most expensive and vulnerable components are subjected to the stresses of the preparation process, which is not desirable in terms of their service life.

Examples of disclosures relating to the radiation devices described above can be found, for example, in U.S. Pat. No. 9,072,572 B2 or US 2022/0202547 A1.

A common feature of many of the radiation devices known from the prior art for use in the dental field is that the generated light emission cannot usually be adapted particularly flexibly to the requirements of the treatment situation, especially with regard to the size of the exposure field, which is usually kept constant in the solutions known from the prior art—or at least cannot be easily adjusted during use.

In the opinion of the inventor, the radiation devices known from the prior art also have the disadvantage that they do not allow efficient, in particular not permanently available, monitoring of the radiation output, in particular not during use in the patient's mouth, which is disadvantageous for efficient quality assurance. Where means for checking the light intensity are provided at all in the state of the art, these are usually designed as separate devices and require the user to perform a separate check step, which can reduce user acceptance of the frequent performance of a regular check and, if necessary, correction of the irradiation—which is desirable in itself.

### SUMMARY

The primary object of the present invention is to eliminate or at least mitigate the disadvantages of the prior art.

In particular, it is the object of the present invention to provide an advantageous radiation device for use in the activation of polymerizable dental materials, which enables a reliable and efficient activation of polymerizable dental materials. It is desirable that the radiation device being specified should have the lowest possible height and enable the reliable application of electromagnetic radiation even in hard-to-reach places in the oral cavity. It is an object of the present invention that the radiation device being specified should have high flexibility for the achievable emission angles and/or the size of the exposure field, wherein the emission angle and the size of the exposure field should ideally be adjustable directly during treatment in order to optimally cure polymerizable dental materials even in particularly difficult-to-access areas and in cavities of different sizes.

It is an object of the present invention that the radiation device being specified should be able to provide the electromagnetic radiation at a high light intensity, wherein it is desirable that a cooling system which may be necessary to realize these high light intensities should be as simple as possible to implement.

In addition, it is an object of the present invention that the radiation device being specified should be particularly robust, in order to achieve a long service life, wherein the essential electronic components should ideally be arranged in such a way that they do not have to be arranged in any exchangeable parts of the radiation device which, after use in the mouth of a patient, have to be subjected to demanding thermal and/or mechanical stresses during preparation.

Furthermore, it is an object of the present invention that the radiation device to be disclosed should be particularly time and/or cost efficient to manufacture, while ideally minimizing the need for complex microelectronics.

Furthermore, it is a supplementary object of the present invention that the radiation device being specified should permit measurement or monitoring of the light intensity actually emitted in a particularly efficient manner, in order to increase the possibility for quality assurance and the user acceptance of quality assurance measures in this respect, wherein it should ideally also be possible to test the light intensity immediately before and/or during the use in activating polymerizable dental materials, in order to ensure a particularly high level of process reliability by means of automated or integrated monitoring of the emitted light intensity—and preferably also by means of efficient measurements immediately during or after.

The inventor of the present invention has found that the above-described objects can be surprisingly achieved in a radiation device by using a mirror which is integrated in the head portion of the housing, provided for insertion into the patient's mouth, and which is oriented in such a manner that the mirror deflects electromagnetic radiation emitted from a radiation source arranged in the base portion, such that the electromagnetic radiation can emerge from the radiation emission window provided therefor.

The corresponding design of the radiation device makes it possible in an advantageous way to arrange the radiation source in the lower base portion of the radiation device, which makes efficient cooling possible. In the part of the radiation device that is inserted into the patient's mouth during use, it is advantageous to be able to dispense almost completely with complex electronics, which means that a particularly robust structure can be obtained, wherein the corresponding head portion is highly durable even with frequent cleaning or sterilization measures, even at high temperatures.

Thanks to the use of a mirror integrated in the housing, a particularly low overall height can be realized, while at the same time high emission angles can be achieved without an undesirable loss of light intensity. The corresponding structure of the radiation device according to the invention can also be manufactured in a particularly time- and cost-efficient manner. Afurther advantage of the radiation device according to the invention is that the mirror can be designed particularly easily as an adjustable mirror, in particular an automatically adjustable mirror, wherein a high degree of flexibility in the deflection of the electromagnetic radiation is achieved with a comparatively simple controller, so that a wide range of emission angles can be set—in particular, also during use in the oral cavity. This allows the light emission to be efficiently directed to hard-to-reach areas in the oral cavity.

Additionally or alternatively, when using a mirror, optical lenses that influence the shape of the beam generated by the radiation source can also be used particularly efficiently. In particular, if the radiation device according to the invention is designed so that the distance between the radiation source and the lens can be adjusted, the size of the exposure field—or, indirectly, the light intensity—can be controlled in a particularly efficient manner.

The inventor considers it to be a particularly great advantage of radiation devices according to the invention that integrated monitoring of the emitted light intensity can be integrated into them particularly easily. By selecting a suitable structural design of the mirror or by implementing an adjustability of the mirror, it is possible to direct the electromagnetic radiation or part of the electromagnetic radiation onto an integrated photosensor, even during use for curing polymerizable dental materials, thereby allowing efficient monitoring of the light intensity.

With regard to the use of a mirror, the invention is roughly based on a concept idea from the late 1980s, as disclosed in EP0360959 A1, which, according to the inventor's knowledge, never became established on the market. In EP 0360959 A1, it was proposed to place a relatively large external mirror with a holder on the light guide of a radiation device, which should deflect the light emerging from the light guide, but also in particular give the dentist a direct view of the irradiated area as an observation mirror. In the inventor's opinion, however, the resulting device was unfavorable in particular due to the unfavorable height and the additional handling effort of a separate component, which increased the cleaning effort. In addition, the inventor believes that the concept of a separate attachment was not suitable for setting precise emission angles so that the actual radiation output depended on the user's manual skills. This could potentially be enough of a problem that an insufficiently mounted mirror could lead to unwanted reflection of radiation so that unwanted radiation exposure with consequential damage could also occur, especially in the case of high-energy radiation. Even though the concept idea at the time also considered the adjustability of the mirror, this had to be done manually and, even assuming precise fastening of the attachment to the beam guide, could not be controlled in a targeted manner. In particular, the mirror could not be adjusted automatically. In particular, the integration of the mirror into the housing of the radiation device, in radiation devices according to the invention, eliminates the existing problems of the concept idea in an advantageous manner, wherein considerable advantages are achieved in an advantageous manner, particularly in the areas of occupational safety, user acceptance, patient comfort, cleanability, durability, automation and sensor integration.

The above-mentioned objects are thus achieved by the subject matter of the invention as defined herein. Preferred embodiments according to the invention are apparent from the following embodiments.

Such embodiments, which are hereinafter designated as preferred, are combined in particularly preferred embodiments with features of other embodiments designated as preferred. Combinations of two or more of the embodiments referred to below as particularly preferred are thus very particularly preferred. Also preferred are embodiments in which a feature of one embodiment designated as preferred to any extent is combined with one or more other features of other embodiments designated as preferred to any extent. Features of preferred methods and uses are derived from the features of preferred radiation devices.

The invention relates in particular to a radiation device for use in activating polymerizable dental materials, comprising:
i) a housing comprising:
i.a) a base portion, and
i.b) a head portion connected to the base portion, the housing comprising a radiation emission window in the head portion,
ii) a radiation source arranged in the region of the base portion in the interior of the housing for emitting electromagnetic radiation, the radiation source having an emission intensity maximum at a wavelength A in the range from 350 to 600 nm, and
iii) a mirror arranged in the region of the head portion in the interior of the housing,
wherein a radiation path extends between the radiation source and the mirror, and the radiation device is configured to allow electromagnetic radiation emitted by the radiation source to arrive at the mirror via the radiation path, and
wherein the mirror is arranged in such a manner that the electromagnetic radiation arriving at the mirror via the radiation path is reflected by the mirror in such a manner that the reflected electromagnetic radiation exits the housing through the radiation emission window.

The radiation device according to the invention is suitable for use in the activation of polymerizable dental materials. This means that it is capable of emitting electromagnetic radiation of a wavelength suitable for curing polymerizable dental materials, in particular in such a way that this radiation can be directed to a cavity in a tooth in order to cause polymerization of a polymerizable dental material there, for example by means of radical polymerization of (meth)acrylate compounds.

Taking into account the intended application in the field of dentistry, the radiation device according to the invention is preferably designed in such a way that it can be held comfortably in one hand and inserted into the patient's mouth without this being perceived as too uncomfortable. Accordingly, a radiation device according to the invention in which the radiation device is a portable radiation device is particularly relevant. An example of this is a radiation device according to the invention in which the radiation device has a length in the range from 10 to 40 cm, preferably in the range from 15 to 35 cm, particularly preferably in the range from 20 to 30 cm. Also exemplary is a radiation device according to the invention in which the radiation device has a weight of less than 500 g, preferably less than 400 g, particularly preferably less than 300 g, most preferably in the range of 100 to 250 g.

A radiation device according to the invention in which the radiation device is an elongated radiation device is typical.

In an advantageous way, the construction of radiation devices according to the invention allows emission angles of greater than 90° to be realized without a curvature so that particularly small construction heights can be realized by a straight or flat design, for example in the manner of a rod. Accordingly, a radiation device according to the invention is also preferred which has an elongate head portion, preferably a straight elongate head portion, particularly preferably a substantially cylindrical head portion. In other words, this means that a radiation device according to the invention in which the head portion is substantially not curved is preferred.

An example of this is an elongate radiation device according to the invention in which the quotient of the length of the radiation device along the longitudinal direction divided by the mean width of the radiation device orthogonal to the longitudinal direction is in the range from 15:1 to 5:1, preferably in the range from 13:1 to 7:1, particularly preferably in the range from 11:1 to 9:1.

Conveniently, the radiation device according to the invention can be optimized by further constructive elements for a pleasant and comfortable use during treatment, for example by providing an optional padded handgrip as well as a partially transparent plastic plate, for example colored, which serves as a screen and protects the eyes of the practitioner from the emitted radiation during the radiation activation of the dental materials. An example of a corresponding radiation device according to the invention is one in which the radiation device comprises a handgrip arranged in the region of the base portion. A further example of a radiation device according to the invention is also exemplary is one in which the radiation device comprises a flat transparent screen, wherein the transparent screen has a reduced transmittance for electromagnetic radiation of the wavelength A, preferably a transmittance of 0.1 or less, preferably 0.05 or less, particularly preferably 0.01 or less, along the thickness direction orthogonal to the surface plane, wherein the screen is preferably attached to the housing in the region of the base portion.

It is conceivable to realize the energy supply for radiation devices according to the invention via an electrical cable. However, with a view to making the use of the corresponding radiation device as user-friendly as possible, the inventor proposes that the radiation device be designed as a cable-free variant. Accordingly, a radiation device according to the invention in which the radiation device comprises an energy storage unit for supplying the radiation device with electrical energy, preferably a battery or an accumulator, preferably a lithium-ion battery, wherein the energy storage unit is preferably arranged around the area of the base portion in the interior of the housing, is preferred.

The radiation device according to the invention initially comprises a housing in or on which the other components of the radiation device are arranged, wherein it is preferable to arrange these in the interior of the housing, particularly in the case of components to which mechanical loads pose a risk. Metal housings not only offer particularly advantageous protective properties, but are also particularly easy to clean. A radiation device according to the invention in which the housing is at least partially, preferably completely, made of plastic or metal, preferably metal, is preferred.

According to the above definition, the housing comprises a base portion and a head portion connected to it. In accordance with professional understanding, this distinguishes different areas of the housing from each other for the purpose of definition, without them necessarily being separate components. As with the dental radiation devices known from the prior art, the head portion is the part of the housing which is intended to be inserted into the patient's mouth during subsequent use, while the base portion refers to the other portions of the housing, in particular the parts which form the grip area of the radiation device—i.e., the part of the housing by which the radiation device is gripped for use and in which, for example, an energy storage unit and/or the control electronics are provided.

Even if, as explained above, it is also possible for a base portion and a head portion to be defined for a one-piece housing, the designation of the portions chosen above already takes account of the fact that it is particularly preferable to design the radiation device and consequently also the housing in several parts. This makes it advantageously possible to separate the head portion, which is inserted into the patient's mouth during use and must subsequently be cleaned or sterilized accordingly, from the base portion after use of the radiation device. Starting from the base portion, which comprises the parts that can be reused even without cleaning, in particular a large part of the necessary electronics, the operational readiness of the radiation device according to the invention can then be restored quickly and efficiently by replacing the head portion. Accordingly, a radiation device according to the invention in which the radiation device and/or the housing of the radiation device is designed in several parts, preferably in two parts, is preferred, wherein a first part of the radiation device comprises the base portion, wherein a second part of the radiation device comprises the head portion, wherein the first part and the second part are reversibly and non-destructively detachably connected to one another. A radiation device according to the invention in which the first part and the second part are reversibly and non-destructively detachably connected to one another via a plug-in connection or a screw connection, preferably a plug-in connection, is preferred.

As explained above, the head portion is intended to be inserted into the patient's mouth to effect the activation of a polymerizable dental material, for example in a cavity. In accordance with the requirements of this application, the head portion comprises a radiation emission window for this purpose, i.e., an opening or transparent area through which electromagnetic radiation can exit from the interior of the radiation device. In practice, this radiation emission window is usually arranged laterally in the housing, for example in the lateral surface of a cylindrical head portion, to achieve the desired high deflection angle of the electromagnetic radiation, and runs, for example, substantially parallel to the longitudinal axis of the radiation device. Against this background, a radiation device according to the invention in which the radiation emission window is arranged at the end of the head portion facing away from the base portion, in particular laterally with respect to the longitudinal direction of the radiation device, is also preferred.

Even if it would be at least theoretically conceivable to realize the necessary radiation emission window by means of a piercing recess in the housing, it is preferable for substantially all embodiments to provide the radiation emission window with a protective cover and to design it accordingly as a transparent area in the housing wall. This makes it possible in an advantageous way to prevent soiling of the optics arranged in the interior of the housing, in particular the mirror. This makes cleaning the radiation device much easier and increases the longevity of the components. In order to achieve the desired high energy efficiency, it is advisable to make the protective screen sufficiently transparent in the relevant radiation range so that it has as little influence as possible on the emitted light intensity. A radiation device according to the invention in which the radiation emission window is at least partially, preferably completely, closed off with a transparent protective pane, preferably made of glass or plastic, is therefore preferred. A radiation device according to the invention in which the transparent protective pane for electromagnetic radiation of wavelength A has a transmittance of 0.95 or more, preferably 0.98 or more, particularly preferably 0.99 or more, along the direction connecting the radiation emission window and the mirror, is preferred.

In the radiation device according to the invention, the electromagnetic radiation, which is to be used later to activate the polymerizable dental materials, is provided by a radiation source.

The above definition of the radiation sources by means of an emission intensity maximum is appropriate in accordance with the understanding of the person skilled in the art, since many radiation sources, for example conventional lamps, can have components in different wavelength ranges in their emission spectrum, which would, however, often not be suitable for efficient activation of corresponding polymerizable dental materials. Rather, in order to activate, for example by the choice of photoinitiators, a radiation-curing dental material which is designed for activation with a wavelength λ_{DM}, it is expedient to select the radiation source such that an emission intensity maximum is at a wavelength A which is sufficiently close to λ_{DM} so that the intensity at λ_{DM} is still sufficiently high, wherein it is particularly energy-efficient and expedient to set λ=λ_{DM} if possible, in accordance with the understanding of those skilled in the art. In light of the above definition, the person skilled in the art will readily understand that, for example, if A is 450 nm, the radiation source is a blue emitter whose emission spectrum includes electromagnetic radiation of a wavelength A of 450 nm—not only as a secondary component, but rather has an emission intensity maximum at this wavelength. In the opinion of the inventor, a radiation device according to the invention is preferred, for the intended use in the dental field, in which the radiation source has an emission intensity maximum at a wavelength A in the range from 380 to 550 nm, preferably in the range from 400 to 500 nm, particularly preferably in the range from 440 to 490 nm, most preferably in the range from 450 to 480 nm.

According to the inventor, LEDs are particularly suitable for use in radiation devices according to the invention, as they can be set with particular precision, especially with regard to the characteristics of the emission spectrum, have a narrow wavelength range, can be operated very energy-efficiently, and also generally require only little installation space. It can be seen as an advantage of radiation devices according to the invention that these can already realize excellent radiation properties with typical LEDs, wherein efficient focusing and low divergence can also be ensured in particular by the use of lenses, as disclosed below. However, the use of laser LEDs can also be advantageous, especially for high-performance applications, for example for activating polymerizable dental materials in particularly deep cavities. Accordingly, a radiation device according to the invention is preferred for substantially all embodiments in which the radiation source is an LED radiation source, preferably a powerful LED radiation source, in particular a laser LED.

A particularly essential component of radiation devices according to the invention is the mirror, which according to the invention is arranged in the interior of the housing, namely in the head portion, and serves to reflect the electromagnetic radiation generated by the radiation source so that it can emerge from the housing via the radiation emission window. Suitable mirrors are commercially available from various manufacturers and can in principle be realized in various configurations, wherein in practice it is particularly important that the mirror provides a reflective surface that is suitable for reflecting the incident radiation without the reflected electromagnetic radiation losing its parallelism beyond a negligible degree—i.e., so that disordered scattering only occurs to an insignificant extent. Such mirrors can, for example, be substrates that are coated with a reflective coating, such as metal.

The person skilled in the art understands that it would be conceivable but not preferred to position a chain of mirrors one behind the other in order to realize the desired deflection, as this increases the constructive design and the susceptibility to errors as well as the risk of intensity losses. Accordingly, a radiation device according to the invention in which the radiation device comprises exactly one or two, preferably exactly one, mirrors in the head portion, and wherein the one or the two mirrors are arranged in such a way that the electromagnetic radiation emerging from the radiation path emerges from the housing only by single or double, preferably only single, reflection through the radiation emission window, is preferred.

The mirrors used in radiation devices according to the invention are mirrors in the narrower sense, wherein the reflection of the electromagnetic radiation takes place at the reflective layer which the mirror has. In the inventor's opinion, plane mirrors or parabolic mirrors are particularly preferable in terms of design. In the context of the present invention, other components which could lead to a deflection of the electromagnetic radiation, in particular as a result of different refractive indices, such as prisms, are not understood as mirrors. In particular, light guides, especially curved light guides as used in the prior art, do not fall within the definition of a mirror as used for the radiation devices according to the invention, in accordance with the understanding of those skilled in the art. The person skilled in the art will readily understand that the use of a mirror in the head portion according to the invention is an advantageous alternative to the use of a light guide. A radiation device according to the invention in which the mirror is a plane mirror or a parabolic mirror, preferably a plane mirror, is preferred. In accordance with a person skilled in the art, a radiation device according to the invention has a mirror which is not an optical fiber and does not comprise an optical fiber.

Owing to the mirror installed in the interior of the housing, the design of radiation devices according to the invention allows high emission angles to be realized in a particularly advantageous way, without having to increase the overall height unnecessarily. To exploit this advantage, a radiation device according to the invention in which the mirror is arranged in such a manner that electromagnetic radiation of the wavelength A arriving at the mirror via the radiation path is deflected at an angle in the range from 60° to 120°, preferably in the range from 70° to 110°, preferably in the range from 80° to 100°, is preferred.

In the above definition, the relative arrangement of the radiation source and the mirror is defined in particular by the arrangement of the respective components in the base portion or in the head portion. In this respect, the person skilled in the art understands that a functional requirement is that the electromagnetic radiation generated by the radiation source can arrive at the mirror and can exit the housing as a result of reflection through the radiation emission window. This necessity is expressed in the above definition by the fact that a radiation path runs between the radiation source and the mirror, via which electromagnetic radiation emitted by the radiation source can reach the mirror. In the activated state of the radiation device, i.e., with the radiation source switched on, the beam path of the electromagnetic radiation will pass via the radiation path and arrive via it at the mirror in order to be reflected there in such a way that the reflected part of the beam path passes through the radiation emission window. Since the radiation source is arranged in the base portion and the mirror in the head portion, it is clear to the person skilled in the art that the radiation path extends through both portions accordingly. In other words, a radiation device according to the invention has a radiation path running partly in the base portion and partly in the head portion.

Even if it is conceivable that the radiation path is accessible from the outside, for example through a recess in the housing, the inventor believes that there is little to be said for such a design. At least theoretically, it would also be possible to realize a radiation path that is not exactly straight, for example through the use of additional mirrors. However, since in the opinion of the inventors there are no reasons for guiding the beam path in this way and, on the contrary, the complexity and vulnerability of the structure would be unnecessarily increased, it is preferable for substantially all embodiments if the radiation path between the radiation source and the mirror runs straight, accordingly enabling direct and unhindered irradiation of the mirror. Accordingly, a radiation device according to the invention with the radiation path running in the interior of the housing is preferred. A radiation device according to the invention is additionally or alternatively preferred in which the radiation path between the radiation source and the mirror is substantially straight.

Even if it were at least theoretically conceivable to realize the radiation path to the mirror largely through a solid transparent medium, for example through a glass or plastic strand, the inventor believes that this would not only be unnecessary but also less preferable in view of the resulting light intensity, and in particular with regard to the resulting weight of the radiation device. Rather, the inventors suggest that a particularly preferred design can be realized if the radiation path is formed as a piercing recess in the interior of the housing, which is filled with air, for example, and accordingly allows the passage of the electromagnetic radiation. In other words, in a radiation device according to the invention, the radiation path is formed by a transparent medium, for example air, wherein the radiation path is preferably formed at least partially by a piercing recess in the interior of the housing.

The design of the radiation path as a piercing recess between the radiation source and the mirror can be implemented particularly efficiently in one-piece radiation devices. In a preferred multi-part design, however, the radiation path also runs in different parts of the radiation device, and would be accessible accordingly when replacing the head portion. In this respect, the above remarks on the usefulness of closing off the radiation emission window apply analogously, since contamination of the radiation path is also to be avoided when the parts of the multi-part radiation device are separated and the components arranged therein, i.e., the radiation device and the mirror, are to be protected. Accordingly, a radiation device according to the invention in which the radiation path—in the case of a multi-part radiation device—comprises one or more transparent sealing disks, preferably made of glass or plastic, wherein the sealing disks are preferably arranged in the connecting region between the first part and the second part, is preferred. A radiation device according to the invention in which the transparent sealing disks have a transmittance for electromagnetic radiation of wavelength A along the direction of the radiation path of 0.95 or more, preferably 0.98 or more, particularly preferably 0.99 or more, is also preferred.

An embodiment of the radiation device according to the invention with a fixed mirror is not only particularly easy to manufacture, but also proves to be particularly robust and error-free in later use. Despite this circumstance, the inventor considers it to be particularly advantageous for substantially all embodiments if the mirror is designed as an adjustable mirror, preferably an automatically adjustable mirror, since this enables particularly efficient manipulation of the emitted beam path, in particular even during use in the patient's mouth. In an advantageous way, the emission angle, i.e., in particular the deflection of the beam path relative to the original emission direction or to the longitudinal axis of the radiation device, can be influenced by the mirror position. If, for example, after aligning the light emission window with the cavity to be irradiated, it is determined that optimal irradiation cannot be achieved with the set emission angle, for example due to difficult accessibility and/or unfavorable alignment of the cavity, this can be adjusted particularly easily, wherein it is also possible in particular to emit the radiation at least partially in the direction counter to the radiation direction originally generated by the radiation source, so that rear-side holes or distal cavities of the anterior teeth in particular can be irradiated particularly efficiently. For substantially all embodiments, a radiation device according to the invention in which the mirror is reversibly and non-destructively movable, preferably reversibly and non-destructively rotatable or displaceable, particularly preferably rotatable, in the interior of the housing, is preferred. A radiation device according to the invention in which the mirror can be rotated by 20° or more, preferably 40° or more, and/or wherein the inclination of the mirror relative to the radiation path can preferably be changed by 20° or more, preferably 40° or more, is preferred. In other words, a radiation device according to the invention in which the mirror is movable in such a way that electromagnetic radiation of wavelength A arriving at the mirror from the radiation path is deflected in an angular range of 80° to 100°, preferably in the angular range of 70° to 110°, particularly preferably in the angular range of 60° to 120°, depending on the position of the mirror, is preferred.

According to the inventors, a particularly good radiation output with advantageous radiation characteristics is achieved if a lens is arranged in the beam path, i.e., is constructed in the radiation path, wherein the dimensions of the lens can be selected by the person skilled in the art in such a way that it influences the radiation emitted by the radiation source in the desired way, for example by focusing it in the direction of the mirror or by orienting or aligning it. Accordingly, a radiation device according to the invention in which the radiation device comprises at least one lens arranged in the radiation path, which is configured to influence, preferably to focus, the electromagnetic radiation emitted by the radiation source before it hits the mirror, is preferred. In a preferred radiation device according to the invention, the lens is arranged in the region of the base portion in the interior of the housing.

A preferred embodiment, which is combined in particularly preferred embodiments in particular with an adjustable mirror and in particular preferably also with a corresponding electronic controller, is obtained if the lens is arranged in the interior of the radiation device in such a way that the distance between the lens and the radiation source can be changed, it being preferred, in the opinion of the inventors, if the lens is designed to be movable in this respect and the radiation source remains substantially stationary. This makes it possible to efficiently adjust the size of the exposure field, especially during use in the oral cavity. The size of the exposure field can be flexibly configured to the requirements of the treatment situation, for example by optimally adapting the size of the exposure field to the size of the cavity to be irradiated. A radiation device according to the invention in which the radiation device is configured so that the distance between the lens and the radiation source can be changed reversibly and non-destructively is preferred. In this respect, a radiation device according to the invention is particularly preferred in which the lens is arranged in the radiation device so as to be reversibly and non-destructively movable, preferably movable relative to the radiation source along the radiation path.

The inventors consider it to be a further particularly advantageous embodiment if the radiation device according to the invention also comprises a photosensor with which the light intensity and/or a parameter correlating with the light intensity, for example a current intensity or voltage registered as a result of irradiation, can be measured. Suitable photosensors are known to the person skilled in the art on the basis of his general knowledge and are commercially available from various manufacturers, wherein the photosensors can be suitably tuned to the respective wavelength of the electromagnetic radiation used. A radiation device according to the invention in which the radiation device comprises at least one photosensor arranged in the interior of the housing, preferably a photodiode, is preferred. This is a radiation device according to the invention, wherein the photosensor is configured to determine intensity information for incoming electromagnetic radiation, wherein the intensity information comprises the intensity of the radiation and/or a parameter correlating with the intensity, in particular the radiant power.

In the opinion of the inventor, it is particularly advantageous to determine the light intensity as near as possible to the exit of the electromagnetic radiation emerging from the radiation device. Accordingly, a radiation device according to the invention, in which the photosensor is arranged in the region of the head portion in the interior of the housing, is preferred.

The person skilled in the art understands that in the structure described above, consisting of a radiation source, mirror and radiation emission window, a beam path is formed during operation, and this should not be interrupted by an inserted photosensor. Accordingly, the inventor has identified constructive embodiments and measures with which a measurement of the radiation intensity during the light application is possible, or can be carried out quickly and efficiently during use, for example at recurring intervals, wherein these solutions synergistically draw on the advantages of using a mirror. In a first embodiment, the mirror arranged between the photosensor and the radiation source can be provided with a recess through which part of the radiation can pass through the mirror onto the photosensor, so that a fraction of the electromagnetic radiation—which in practice is minimized by using a very small hole—is continuously available for measuring the radiation intensity. A radiation device according to the invention in which the mirror is arranged between the photosensor and the radiation source is preferred for this purpose. Preferred in this respect is a radiation device according to the invention in which the mirror comprises a piercing recess, wherein the radiation device is designed such that a portion of the electromagnetic radiation of wavelength A arriving at the mirror via the radiation path is directed through the piercing recess onto the photosensor.

In the opinion of the inventor, the embodiment described above with a recess in the mirror is particularly useful when using a stationary, i.e., non-adjustable mirror, but can lead to an interference point in the exposure field of the radiation device due to the hole in the mirror, in which the intensity is reduced due to the lack of reflection. In this respect, the inventor proposes as particularly advantageous that the already preferred adjustable version of the mirror can be used to carry out a measurement as required, for example immediately after activation of the radiation source. Two basic concepts here are that the mirror can either be adjusted so that it temporarily does not block the radiation path between the radiation source and the photosensor, or so that the mirror temporarily does not reflect the electromagnetic radiation through the radiation emission window, but rather reflects the radiation back onto the photosensor, in which it can then be measured. A particularly preferred radiation device according to the invention has a radiation device designed such that the position of the mirror and/or the orientation of the mirror, preferably the orientation of the mirror, in particular the rotational position, can be changed reversibly and non-destructively, in such a manner that
x) the electromagnetic radiation arriving at the mirror from the radiation path is reflected in such a way that the reflected electromagnetic radiation is directed onto the photosensor, or
y) the electromagnetic radiation is directed past the mirror onto the photosensor.

In particular in embodiments of radiation devices according to the invention in which the central components are largely stationary and/or are rotationally fixed, in particular using a stationary mirror in conjunction with a stationary lens, the necessary control electronics can be kept very simple in an advantageous manner, which in turn results in particularly robust radiation devices that can also be produced in a particularly cost-efficient manner. In the opinion of the inventor, however, it is preferable for the vast majority of radiation devices according to the invention to provide an electronic data processing device in them which is configured as a control device to control the radiation device according to the invention, and, in particular, to enable the functionalities described above which result from reversibly and non-destructively movable components. Corresponding computing units or processors suitable for this purpose, as well as the necessary control software, are commercially available from various suppliers and/or can be configured to the requirements of the specialist without major effort. A radiation device according to the invention in which the radiation device comprises an electronic data processing device for controlling and/or regulating the radiation device, wherein the electronic data processing device is preferably arranged in the region of the base portion in the interior of the housing, is preferred for substantially all embodiments.

The preferably-used electronic data processing device is ideally configured to control the functionalities described above as preferred as well as the general radiation power, for example automatically, in response to a sensor value, in particular of the photosensor, or in response to an input from the user. A radiation device according to the invention is thus initially preferred in which the electronic data processing device is configured to change the radiation output of the radiation source reversibly and non-destructively, preferably as a result of a manual input and/or as a result of intensity information determined by the photosensor. Preferred is additionally or alternatively a radiation device according to the invention in which the electronic data processing device is configured to change the position of the mirror, in particular the rotational position, and/or the orientation of the mirror, reversibly and non-destructively, preferably as a result of a manual input, in order to change the deflection angle of the reflected radiation, preferably in an angular range of 80° to 100°, particularly preferably in the angular range of 70⁰ to 110°, very particularly preferably in the angular range of 60° to 120°. Also preferred, additionally or alternatively, is a radiation device according to the invention in which the electronic data processing device is configured to change the position of the lens relative to the radiation source along the radiation path reversibly and non-destructively, preferably as a result of a manual input and/or as a result of intensity information determined by the photosensor.

Furthermore, in addition or as an alternative to the embodiments disclosed above, a radiation device according to the invention is preferred in which the electronic data processing device is configured to change the position of the mirror, in particular the rotational position, and/or the movement of the mirror, preferably the rotational position of the mirror, reversibly and non-destructively, in such a way that
x) the electromagnetic radiation arriving at the mirror from the radiation path is reflected in such a way that the reflected electromagnetic radiation is directed onto the photosensor, or
y) the electromagnetic radiation is directed past the mirror via the radiation path to the photosensor.

If a photosensor is used, a radiation device according to the invention in which the radiation device is configured to determine intensity information for the electromagnetic radiation emitted by the radiation source in the interior of the housing with the photosensor during operation, continuously or as a result of a predetermined trigger condition, preferably at a predetermined time after activation or as a result of manual input, is preferred.

If a photosensor is used, the embodiment described below for the method according to the invention is preferred—namely, a radiation device according to the invention, wherein the electronic data processing device is configured to control or regulate the power of the radiation source and/or the distance between the radiation source and the lens as a function of the intensity information.

To increase operational safety, the electronic data processing device can also be designed to implement protective or corrective measures if the intensity measured by the photosensor deviates from the specified intensity, for example because it is too low or too high. A radiation device according to the invention in which the electronic data processing device is configured to compare intensity information determined by the photosensor with a predetermined reference criterion, wherein the electronic data processing device is configured to initiate measures if the deviation from the reference criterion is outside a predetermined tolerance range, is preferred for this purpose. A radiation device according to the invention in which the measures are selected from the group consisting of the deactivation of the radiation device and the output of a warning signal, preferably an optical and/or acoustic warning signal, is preferred.

It can be seen as an advantage of radiation devices according to the invention that they can be designed particularly efficiently for high radiation outputs and light intensities, which can also be maintained for relatively long periods of time. Accordingly, it is also preferable to make use of this suitability to achieve high intensities in radiation devices according to the invention. In particular, a radiation device according to the invention, in which the radiation device is configured to emit electromagnetic radiation from the radiation emission window with an intensity of 500 mW/cm² or more, preferably 800 mW/cm² or more, particularly preferably 1000 mW/cm² or more, preferably for a time of 10 s or more, particularly preferably 15 s or more, is preferred.

In particular, when high intensities and/or long exposure times are used, the inventor believes that it is preferable to also provide elements for dissipating heat from the radiation source, wherein these can be installed particularly easily and efficiently in radiation devices according to the invention thanks to the arrangement of the radiation source in the base portion. A radiation device according to the invention is thus preferred—in particular for powerful radiation devices—in which the radiation device comprises at least one element for dissipating heat from the radiation source, preferably a passive cooling element.

The invention also relates to a method for operating a radiation device according to the invention, comprising the method steps of:
a) generating electromagnetic radiation using the radiation source, the electromagnetic radiation having an intensity maximum at a wavelength A in the range from 350 to 600 nm,
b) guiding the electromagnetic radiation in the interior of the housing from the radiation source to the mirror via the radiation path, and
c) deflecting the electromagnetic radiation using the mirror so that the reflected electromagnetic radiation exits the housing through the radiation emission window.

Preferred embodiments of the method according to the invention are those in which the preferred radiation devices according to the invention disclosed above are used, and also are utilized with regard to the respective advantageous functionalities, for example for the implementation of high radiation output, the changeability of the exit angle from the radiation emission window, or the adaptability of the exposure field.

A method according to the invention is thus initially preferred in which the electromagnetic radiation emerging from the radiation emission window has an intensity of 500 mW/cm² or more, preferably of 800 mW/cm² or more, particularly preferably of 1000 mW/cm² or more.

Preferred is additionally or alternatively also a method according to the invention in which the radiation device is operated for a time of 10 s or more, preferably 15 s or more, particularly preferably 20 s or more.

Additionally or alternatively, a method according to the invention in which the position of the mirror is changed in order to change the deflection of the electromagnetic radiation and the exit angle from the radiation emission window, preferably as a function of the shape and position of the substrate to be irradiated, is preferred.

Furthermore, a method according to the invention is additionally or alternatively preferred in which the distance between the radiation source and the lens is changed in order to adjust the size of the radiation cross-section and/or the power density of the electromagnetic radiation emerging from the radiation emission window, preferably as a function of the nature of the substrate to be irradiated and/or the material properties of the material to be irradiated.

A method according to the invention is again additionally or alternatively preferred in which the method additionally comprises one of the following method steps, which are carried out at time intervals, preferably at predetermined time intervals:
d1) temporarily changing the position of the mirror and/or the rotational position of the mirror so that the electromagnetic radiation is reflected, such that the reflected electromagnetic radiation is directed to the photosensor, or
d2) temporarily changing the position of the mirror and/or the rotational position of the mirror so that the electromagnetic radiation is directed past the mirror onto the photosensor.

Additionally or alternatively, a method according to the invention in which the power of the radiation source and/or the distance between the radiation source and the lens are controlled or regulated as a function of the intensity information is preferred.

Furthermore, a method according to the invention is additionally or alternatively preferred in which the intensity information determined by the photosensor is compared with a predetermined reference criterion, wherein measures are initiated if the deviation from the reference criterion is outside a predetermined tolerance range.

Also disclosed is the use of a radiation device according to the invention for activating polymerizable dental materials in the production and/or processing of dental restorations.

During the development of the invention, it has been shown that the use of an integrated photosensor disclosed above for radiation devices according to the invention is also advantageous independently of the use of a mirror according to the invention. Based on this finding, a generally advantageous embodiment of a radiation device is disclosed below in addition to or as an alternative to the radiation devices according to the invention. To clearly distinguish it from radiation devices according to the invention, this embodiment is hereinafter referred to as a “dental light device.”

Disclosed, in addition or alternatively to radiation devices according to the invention, is correspondingly a dental light device for use in activating polymerizable dental materials, comprising:
i-2) a housing comprising an interior with a radiation outlet, preferably with a radiation emission window,
ii-2) a radiation source arranged in the interior of the housing for emitting electromagnetic radiation, the radiation source having an emission intensity maximum at a wavelength A in the range from 350 to 600 nm,
iii-2) a photosensor arranged in the interior of the housing, preferably a photodiode, and
iv-2) an electronic data processing device,
wherein the dental light device is configured to determine intensity information for the electromagnetic radiation emitted by the radiation source during operation, continuously or as a result of a predetermined trigger condition, preferably at a predetermined time after activation or as a result of manual input, in the interior of the housing, using the photosensor,
wherein the electronic data processing device is arranged to control and/or regulate the dental light device as a function of the determined intensity information.

Particularly preferred in this case are designs of the dental light device in which the intensity information is determined as a result of a predetermined triggering condition. Preferred is additionally or alternatively a dental light device, wherein the dental light device comprises a movable mirror so that the electromagnetic radiation can be deflected onto the photosensor as a result of a predetermined trigger condition and/or wherein the photosensor is reversibly and non-destructively movably arranged in the interior so that the photosensor can be brought into the beam path of the electromagnetic radiation as a result of a predetermined trigger condition.

Also disclosed is a method of operating a dental light device, comprising the steps of:
aa) generating electromagnetic radiation using the radiation source, the electromagnetic radiation having an intensity maximum at a wavelength A in the range from 350 to 600 nm, and
bb) guiding the electromagnetic radiation in the interior of the housing to the radiation outlet,
wherein the photosensor arranged in the interior is used to determine intensity information, continuously during operation or as a result of a predetermined trigger condition, preferably at a predetermined time after activation or as a result of manual input, using the photosensor arranged in the interior, and
wherein the dental light device is controlled and/or regulated as a function of the determined intensity information.

Features of preferred dental light devices and methods result from the features of radiation devices and methods according to the invention, wherein the explanations of the corresponding advantages apply accordingly.

### BRIEF DESCRIPTION OF THE DRAWINGS

The invention and preferred embodiments of the invention are explained and described in more detail below with reference to accompanying figures. In the figures:

FIG. 1 shows a schematic cross-sectional representation of a radiation device according to the invention in a preferred embodiment;

FIG. 2 shows a schematic visualization of the adjustability of the mirror in radiation devices according to the invention in preferred embodiments; and

FIG. 3 shows a schematic representation for the realization of the irradiation of a photosensor in radiation devices according to the invention, in a preferred embodiment.

### DETAILED DESCRIPTION

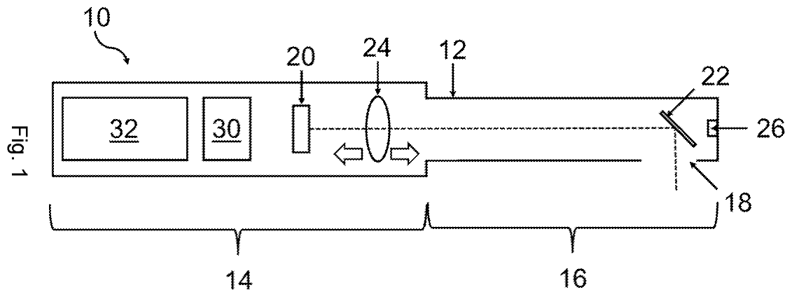

FIG. 1 shows a schematic cross-sectional view of a radiation device 10 according to the invention which is suitable for use in activating polymerizable dental materials in the oral cavity. The radiation device 10 comprises a housing 12 in which the components of the radiation device 10 are arranged. The housing 12 comprises a base portion 14 and a head portion 16, each of which is indicated by curly brackets.

The radiation device 10 of FIG. 1 is designed in two parts, wherein the lower part forming the base portion 14, and the head portion 16 intended for insertion into the mouth, are reversibly and non-destructively detachably connected to each other at the interface by a plug-in connection (not shown).

A radiation source 20 is arranged in the interior of the housing 12, whose emission intensity maximum in the example shown is at a wavelength A of 450 nm. The radiation device 10 is designed so that the electromagnetic radiation generated by the radiation source 20 can arrive as a straight beam via the radiation path onto the mirror 22, which is arranged in the head portion 16. The radiation path is substantially implemented as an air-filled recess in the interior of the housing 12, which is closed off only in the contact area of the two parts of the housing with a substantially transparent glass pane (not shown).

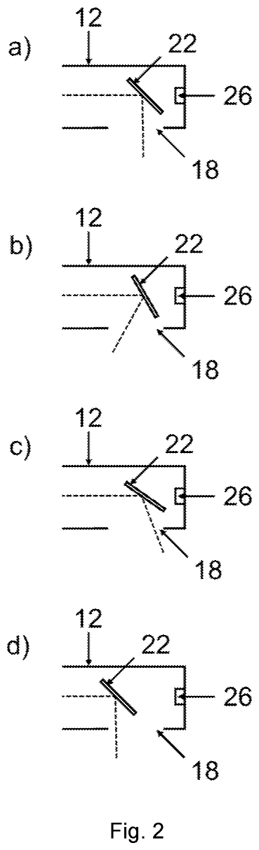

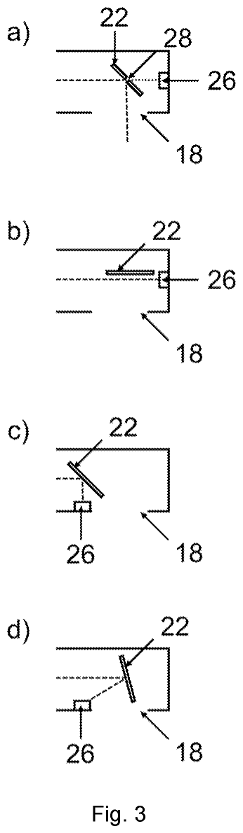

FIG. 1 shows how the electromagnetic radiation generated by the radiation source 20, which is shown as a dashed line, is reflected and deflected by the mirror 22 at approximately 90° so that the deflected electromagnetic radiation can exit the housing 12 through the radiation emission window 18. In the preferred embodiment of FIG. 1, a lens 24 is also arranged in the radiation path—i.e., when activated in the beam path of the electromagnetic radiation, it can be reversibly and non-destructively displaced along the beam path in order to influence the electromagnetic radiation emitted by the radiation source 20 in such a way that the size of the exposure field can be adjusted as a result. In addition to the radiation source 20 and the lens 24, an electronic data processing device 30 and an energy storage unit 32 are also arranged in the interior of the housing in the base portion 14, the latter serving to supply the radiation device 10 according to the invention with electrical energy. In the example shown, the electronic data processing device 30 is set up not only to control the radiation power of the radiation source 20, but also to change the position of the lens 24, in particular as a result of input from a user. In addition, the electronic data processing device 30 is configured to control the adjustable mirror 22, thereby in particular changing the emission angle formed by the radiation emission window 18 and the irradiation of the photosensor 26, as shown schematically in FIGS. 2 and 3.

FIG. 2 shows, in four simplified enlargements of the head portion 16 of the radiation device 10 according to the invention of FIG. 1, different positionings of the mirror 22, which can be adjusted via the electronic data processing device 30. FIG. 2a) corresponds to the orientation shown in FIG. 1. In FIG. 2b) and FIG. 2c), the position of the mirror is changed by tilting it in order to make the emission angle lower or higher, which makes it possible to adapt to the respective operating conditions in an advantageous way. In contrast, in FIG. 2d) there is no tilting, but rather a translational movement of the mirror 22 along the radiation path. This makes it advantageously possible to fine-tune the irradiated position without having to move the radiation device 10 as a whole.

FIG. 3 again shows in simplified portional views of the head portion 16 various possibilities for realizing a measurement of the radiation intensity of the electromagnetic radiation with the photosensor 26. In FIG. 3a), the mirror 22 is equipped with a piercing recess 28 for this purpose, through which a portion of the electromagnetic radiation can pass through the mirror 22 and arrive at the photosensor 26 in order to be measured there. FIG. 3b) shows an embodiment in which the adjustability of the mirror, in particular the pivotability of the mirror, is utilized in order to move it out of the beam path connecting the radiation source 20 and the photosensor 26—as required, for example, to determine the radiation intensity as a result of a quality assurance operation triggered by the user.

In contrast, FIGS. 3c) and 3d) show embodiments in which the mirror 22 is used to reflect the electromagnetic radiation onto the photosensor 26. In FIG. 3c), this is achieved by sliding the mirror 22 along the radiation path in order to apply radiation to a photosensor 26 arranged next to the radiation emission window 18. In FIG. 3d), on the other hand, a rotational adjustment of the mirror 22 is utilized in order to temporarily no longer direct the reflected electromagnetic radiation out of the housing 12 through the radiation emission window 18, but rather onto the photosensor 26.

### LIST OF REFERENCE SYMBOLS

10 Radiation device
12 Housing
14 Base portion
16 Head portion
18 Radiation emission window
20 Radiation source
22 Mirror
24 Lens
26 Photosensor
28 Recess
30 Data processing device
32 Energy storage unit

1. A radiation device for use in activating polymerizable dental materials, comprising:
a housing comprising:
a base portion, and
a head portion connected to the base portion, wherein the housing comprises a radiation emission window in the head portion,
a radiation source arranged in the region of the base portion in the interior of the housing, for emitting electromagnetic radiation, the radiation source having an emission intensity maximum at a wavelength in a range from 350 to 600 nm, and
a mirror arranged in the region of the head portion in the interior of the housing,
wherein a radiation path extends between the radiation source and the mirror, and the radiation device is configured to allow electromagnetic radiation emitted by the radiation source to arrive at the mirror via the radiation path, and
wherein the mirror is arranged such that the electromagnetic radiation arriving at the mirror via the radiation path is reflected by the mirror such that the reflected electromagnetic radiation exits the housing through the radiation emission window.

2. The radiation device according to claim 1, wherein the radiation source has an emission intensity maximum at a wavelength in a range from 380 to 550 nm.

3. The radiation device according to claim 1, wherein the radiation device comprises at least one lens arranged in the radiation path and configured to influence the electromagnetic radiation emitted by the radiation source before it arrives at the mirror.

4. The radiation device according to claim 3, wherein the radiation device is configured so that a distance between the lens and the radiation source is reversibly and non-destructively changeable.

5. The radiation device according to claim 1, wherein the mirror is reversibly and non-destructively movably arranged in the interior of the housing.

6. The radiation device according to claim 1, wherein the radiation device comprises at least one photosensor.

7. The radiation device according to claim 6, wherein the mirror is arranged between the photosensor and the radiation source.

8. The radiation device according to claim 6, wherein the radiation device is configured such that one or more of a position of the mirror and an orientation of the mirror is reversibly and non-destructively changeable such that:
the electromagnetic radiation arriving at the mirror via the radiation path is reflected in such a way that the reflected electromagnetic radiation is directed onto the photosensor, or
the electromagnetic radiation is directed past the mirror onto the photosensor.

9. The radiation device according to claim 7, wherein the mirror comprises a piercing recess, wherein the radiation device is configured such that a portion of the electromagnetic radiation of wavelength arriving at the mirror via the radiation path is directed through the piercing recess onto the photosensor.

10. The radiation device according to claim 6, wherein the photosensor is arranged to determine intensity information for incoming electromagnetic radiation, wherein the intensity information comprises one or more of an intensity of the radiation and a parameter correlating with the intensity.

11. The radiation device according to claim 10, further comprising an electronic data processing device for one or more of controlling and regulating the radiation device, wherein the electronic data processing device is arranged to control one or more of power of the radiation source and distance between the radiation source and the lens in accordance with the intensity information.

12. The radiation device according to claim 10, wherein the electronic data processing device is configured to reversibly and non-destructively change the position of the lens relative to the radiation source along the radiation path.

13. The radiation device according to claim 1, wherein the electronic data processing device is configured to reversibly and non-destructively change the position and/or orientation of the mirror.

14. The radiation device according to claim 1, wherein the radiation device is arranged to emit electromagnetic radiation with an intensity of at least 500 mW/cm² from the radiation emission window.

15. A method for operating a radiation device according to claim 1, comprising:
generating electromagnetic radiation using the radiation source, the electromagnetic radiation having an intensity maximum at a wavelength in the range from 350 to 600 nm,
guiding the electromagnetic radiation in the interior of the housing from the radiation source to the mirror via the radiation path, and
deflecting the electromagnetic radiation using the mirror so that the reflected electromagnetic radiation exits the housing through the radiation emission window.

16. The radiation device according to claim 7, wherein the radiation device is configured such that one or more of a position of the mirror and an orientation of the mirror is reversibly and non-destructively changeable such that:
the electromagnetic radiation arriving at the mirror via the radiation path is reflected in such a way that the reflected electromagnetic radiation is directed onto the photosensor, or
the electromagnetic radiation is directed past the mirror onto the photosensor.

17. The radiation device according to claim 7, wherein the photosensor is arranged to determine intensity information for incoming electromagnetic radiation, wherein the intensity information comprises one or more of an intensity of the radiation and a parameter correlating with the intensity.

18. The radiation device according to claim 8, wherein the photosensor is arranged to determine intensity information for incoming electromagnetic radiation, wherein the intensity information comprises one or more of an intensity of the radiation and a parameter correlating with the intensity.

19. The radiation device according to claim 9, wherein the photosensor is arranged to determine intensity information for incoming electromagnetic radiation, wherein the intensity information comprises one or more of an intensity of the radiation and a parameter correlating with the intensity.
